(19) United States
(12) Patent Application Publication
(10) Pub. No.: US 2025/0344940 A1
(43) Pub. Date: Nov. 13, 2025

(54) ULTRASONIC ENDOSCOPE

(71) Applicant: FUJIFILM Corporation, Tokyo (JP)

(72) Inventors: Karin HATTORI, Kanagawa (JP); Masaya INOUE, Kanagawa (JP); Yasuhiko MORIMOTO, Kanagawa (JP)

(73) Assignee: FUJIFILM Corporation, Tokyo (JP)

(21) Appl. No.: 19/189,280

(22) Filed: Apr. 25, 2025

(30) Foreign Application Priority Data
May 10, 2024 (JP) 2024-077273

(51) Int. Cl. A61B 1/00; A61B 1/06

(52) U.S. Cl. CPC A61B 1/00098; A61B 1/00091; A61B 1/00096; A61B 1/00137; A61B 1/0676

(57) ABSTRACT

An ultrasonic endoscope includes: a distal end portion; an elevator housing portion disposed at the distal end portion and having an opening on a Z(+) direction side in a Z direction orthogonal to a longitudinal axis direction, and configured to house an elevator therein; and a disposition surface that is disposed on a base end side in the longitudinal axis direction with respect to the elevator housing portion, includes a normal component toward a distal end side in the longitudinal axis direction, and is provided with a supply nozzle and an observation window disposed thereon. When viewed from the Z(+) direction side in the Z direction, at least a part of a region of the supply nozzle is disposed at a position overlapping with a base end side region in which the opening of the elevator housing portion is extended to the base end side in the longitudinal axis direction.

### CROSS-REFERENCE TO RELATED APPLICATION

The present application claims priority under 35 U.S.C § 119(a) to Japanese Patent Application No. 2024-077273 filed on May 10, 2024, which is hereby expressly incorporated by reference, in its entirety, into the present application.

### BACKGROUND OF THE INVENTION

### 1. Field of the Invention

The present invention relates to an ultrasonic endoscope, and more particularly to an ultrasonic endoscope comprising an ultrasonic transducer provided at a distal end portion of an insertion part.

### 2. Description of the Related Art

As the ultrasonic endoscope, an ultrasonic endoscope is known which comprises a convex type ultrasonic transducer at a distal end portion of an insertion part, in which a treatment tool outlet port is disposed on a base end side of the ultrasonic transducer at the distal end portion (for example, see WO2020/179909A).

In an examination using the above-described ultrasonic endoscope, for example, while acquiring an ultrasonic image of a treatment target part by an ultrasonic transducer, a biopsy needle (treatment tool) led out into a body through a treatment tool insertion channel and a treatment tool outlet port is punctured into the treatment target part to collect cells. In this case, the treatment tool is treated at a desired position by changing a leading-out direction by an elevating and falling operation of an elevator housed in an elevator housing portion.

The distal end portion of the insertion part is provided with an observation window for observing a treatment target part, an illumination window for emitting illumination light toward the treatment target part, and an air and water supply nozzle for ejecting cleaning water or air toward the observation window or the like, in addition to the ultrasonic transducer and the elevator.

### SUMMARY OF THE INVENTION

In the field of medical endoscopes, it is desired to reduce the diameter (perform thinning) of the distal end portion of the insertion part from the viewpoint of reducing a burden on a patient or reducing complications. However, the ultrasonic endoscope has a problem in that it is difficult to reduce the diameter of the distal end portion because the ultrasonic endoscope has many functions compared to other endoscopes (for example, an endoscope for a digestive system).

The present invention has been made in view of such circumstances, and an object of the present invention is to provide an ultrasonic endoscope that can achieve reduction in diameter of a distal end portion of an insertion part.

A first aspect of the present invention relates to an ultrasonic endoscope comprising: a distal end portion that is disposed on a distal end side of an insertion part extending along a longitudinal axis direction; an elevator housing portion that is disposed at the distal end portion, that has an opening on one side in a first direction orthogonal to the longitudinal axis direction, and that houses an elevator therein; and a disposition surface that is disposed on a base end side in the longitudinal axis direction with respect to the elevator housing portion, that includes a normal component toward a distal end side in the longitudinal axis direction, and that is provided with a fluid nozzle and an observation window disposed thereon, in which, in a case of being viewed from the one side in the first direction, at least a part of a region of the fluid nozzle is disposed at a position overlapping with a base end side region in which the opening of the elevator housing portion is extended to the base end side in the longitudinal axis direction.

A second aspect of the present invention relates to the ultrasonic endoscope according to the first aspect, in which the observation window is disposed outside the base end side region.

A third aspect of the present invention relates to the ultrasonic endoscope according to the second aspect, in which the observation window is disposed at a position facing an ejection port side of the fluid nozzle.

A fourth aspect of the present invention relates to the ultrasonic endoscope according to any one of claims the first to third aspects, in which the disposition surface is provided with a first illumination window disposed outside the base end side region.

A fifth aspect of the present invention relates to the ultrasonic endoscope according to the fourth aspect, in which the first illumination window is disposed on an opposite side to a side on which the fluid nozzle is disposed with respect to the observation window.

A sixth aspect of the present invention relates to the ultrasonic endoscope according to any one of the first to fifth aspects, in which the disposition surface is provided with a second illumination window of which at least a part of a region is disposed outside the base end side region.

A seventh aspect of the present invention relates to the ultrasonic endoscope according to the sixth aspect, in which the second illumination window is disposed on an opposite side to a side on which the observation window is disposed with respect to the fluid nozzle.

An eighth aspect of the present invention relates to the ultrasonic endoscope according to any one of the first to seventh aspects, in which the disposition surface includes a first surface region in which the fluid nozzle is disposed and a second surface region in which the observation window is disposed, and in a case in which a normal direction of the disposition surface is defined as a height direction, a height of the first surface region is lower than a height of the second surface region.

A ninth aspect of the present invention relates to the ultrasonic endoscope according to any one of the first to eighth aspects, further comprising: a cover member that is provided at the distal end portion and that is disposed on one side of the fluid nozzle in the first direction.

A tenth aspect of the present invention relates to the ultrasonic endoscope according to the ninth aspect, in which the cover member is made of a material that is the same as a material of the distal end portion.

An eleventh aspect of the present invention relates to the ultrasonic endoscope according to the ninth or tenth aspect, in which the cover member covers an outermost end portion side of the fluid nozzle on the one side in the first direction.

A twelfth aspect of the present invention relates to the ultrasonic endoscope according to any one of the ninth to eleventh aspects, further comprising: a first filler that fills a gap between the fluid nozzle and the cover member.

A thirteenth aspect of the present invention relates to the ultrasonic endoscope according to any one of the first to eighth aspects, in which, in a case of being viewed from an ejection port side of the fluid nozzle, an outermost end portion side of the fluid nozzle on the one side in the first direction is formed in an R-shape.

A fourteenth aspect of the present invention relates to the ultrasonic endoscope according to any one of the first to eighth aspects, further comprising: a second filler that fills a level difference formed on one side of the fluid nozzle in the first direction.

A fifteenth aspect of the present invention relates to the ultrasonic endoscope according to any one of the first to fourteenth aspects, in which the disposition surface includes a guide surface that is provided on an opposite side to a side on which the fluid nozzle is provided with respect to the observation window and that guides fluid ejected from the fluid nozzle toward the elevator housing portion.

A sixteenth aspect of the present invention relates to the ultrasonic endoscope according to the fifteenth aspect, in which a guide groove that guides the fluid guided by the guide surface to an inside of the elevator housing portion is provided at an edge portion of the opening of the elevator housing portion.

According to the aspects of the present invention, it is possible to reduce the diameter of the distal end portion of the insertion part.

### BRIEF DESCRIPTION OF THE DRAWINGS

FIG. 1 is an overall configuration diagram of an ultrasonic endoscope according to the present embodiment.

FIG. 2 is a bird's-eye view of a front surface side of a distal end member in a case of being viewed obliquely from above.

FIG. 3 is a bird's-eye view of a left surface side of the distal end member in a case of being viewed obliquely from above.

FIG. 4 is an explanatory diagram schematically showing disposition positions of an observation window, an illumination window, and an air and water supply nozzle in a case of being viewed from a Z(+) direction side.

FIG. 5 is an explanatory diagram showing disposition positions of the observation window, the illumination window, and the air and water supply nozzle in a case of being viewed from a Y(+) direction side.

FIG. 6 is an explanatory diagram schematically showing the respective disposition positions of the observation window, the illumination window, and the air and water supply nozzle in a case of being viewed from the Z(+) direction side.

FIG. 7 is an enlarged view of the air and water supply nozzle in a case of being viewed from an X(+) direction side.

FIG. 8 is an enlarged view of the air and water supply nozzle in a case of being viewed from an X(−) direction side.

FIG. 9 is an enlarged view of the air and water supply nozzle in a case of being viewed from the X(+) direction side.

FIG. 10 is a bird's-eye view of the left surface side of the distal end member in a case of being viewed obliquely from above.

### DESCRIPTION OF THE PREFERRED EMBODIMENTS

Hereinafter, an embodiment of the present invention will be described with reference to the accompanying drawings.

### Overall Configuration of Ultrasonic Endoscope

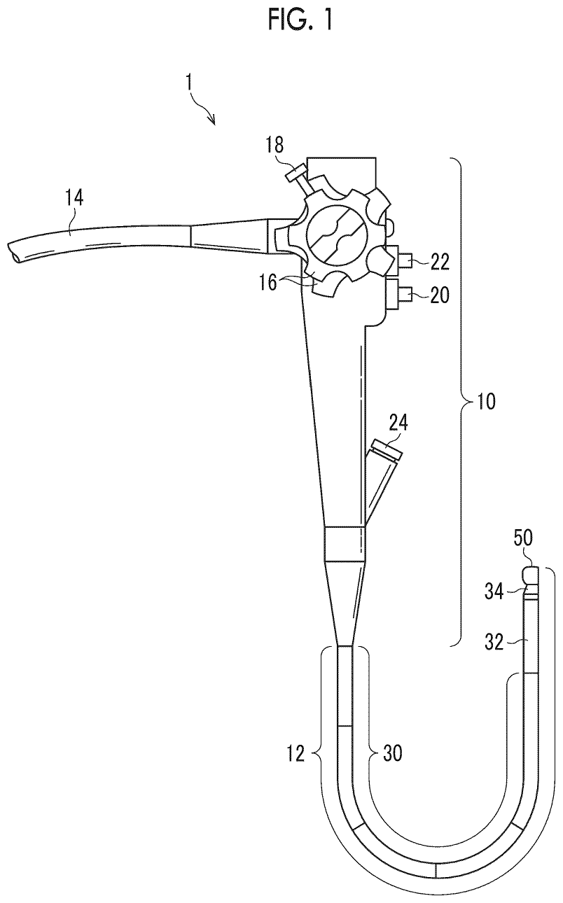

FIG. 1 is an overall diagram of an ultrasonic endoscope (hereinafter, abbreviated as an “endoscope”) 1 according to the embodiment of the present invention.

As shown in FIG. 1, the endoscope 1 according to the present embodiment comprises an operating part 10 that is gripped by an operator to perform various operations, an insertion part 12 that is inserted into a body cavity of a patient, and a universal cord 14. The endoscope 1 is connected to a system constituent device including a processor device and a light source device (not shown) through the universal cord 14.

The operating part 10 is provided with various operation members operated by the operator and, for example, is provided with a pair of angle knobs 16, an elevating operation lever 18, an air and water supply button 20, a suction button 22, and the like.

In addition, a treatment tool inlet port 24 is provided on a distal end side of the operating part 10. The treatment tool introduced from the treatment tool inlet port 24 is inserted into a treatment tool insertion channel inserted into the insertion part 12.

The insertion part 12 extends from a distal end of the operating part 10 along a longitudinal axis direction, and the entire insertion part 12 is formed to have a small diameter and a long shape. The insertion part 12 includes a soft portion 30, a bendable portion 32, and a distal end portion 34 in order from a base end side to a distal end side. It should be noted that the insertion part 12 is an example of an insertion part according to the embodiment of the present invention, and the distal end portion 34 is an example of a distal end portion according to the embodiment of the present invention.

The soft portion 30 occupies most of the insertion part 12 from the base end side and has enough flexibility to be bent in any direction. In a case in which the insertion part 12 is inserted into the body cavity, the soft portion 30 is bent along an insertion route in the body cavity.

The bendable portion 32 is bent in an up-down direction and a left-right direction by a rotational movement operation of the pair of angle knobs 16 of the operating part 10. The distal end portion 34 can be directed in a desired direction by performing the bending operation of the bendable portion 32.

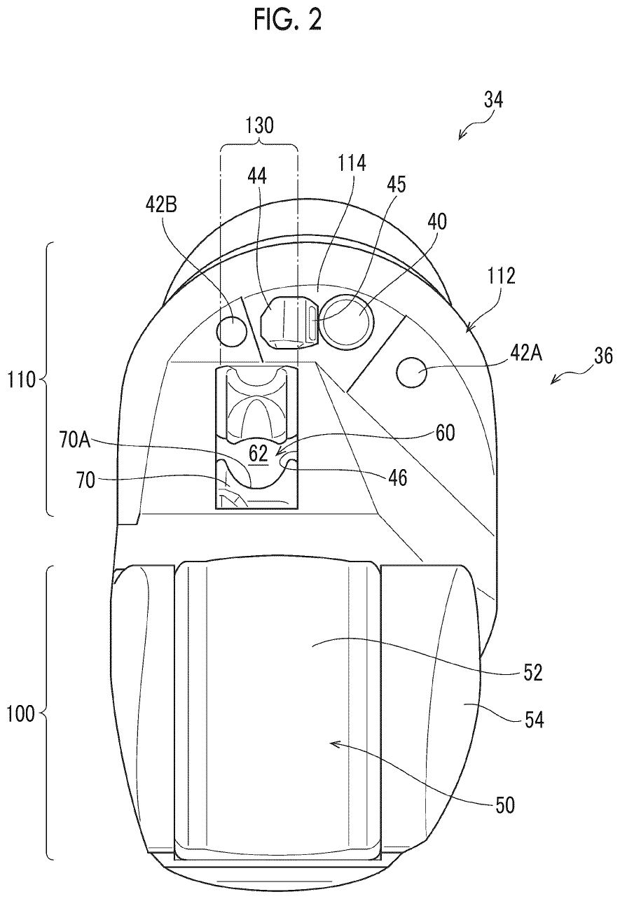

The distal end portion 34 comprises a distal end member 36 (see FIG. 2) described later. The distal end member 36 includes an ultrasonic observation portion 100 and an endoscope observation portion 110.

The universal cord 14 shown in FIG. 1 encompasses built-in components such as an ultrasonic cable, an electric cable, a light guide, and a fluid tube. A connector is provided at an end portion of the universal cord 14 (not shown), and by connecting the connector to the system constituent device, a control signal, power, illumination light, liquid, gas, and the like necessary for the operation of the endoscope 1 are supplied from the system constituent device to the endoscope 1. It should be noted that, in addition to the built-in components, a built-in component such as a treatment tool insertion channel, a bending operation wire, and an elevator operation wire is inserted into the insertion part 12.

Data of an ultrasonic image and data of an endoscopic image acquired by the endoscope 1 (ultrasonic observation portion 100 and endoscope observation portion 110) are each transmitted from the endoscope 1 to the system constituent device. Each data transmitted to the system constituent device is processed by the system constituent device and then displayed on a monitor (not shown) as the ultrasonic image and the endoscopic image, respectively.

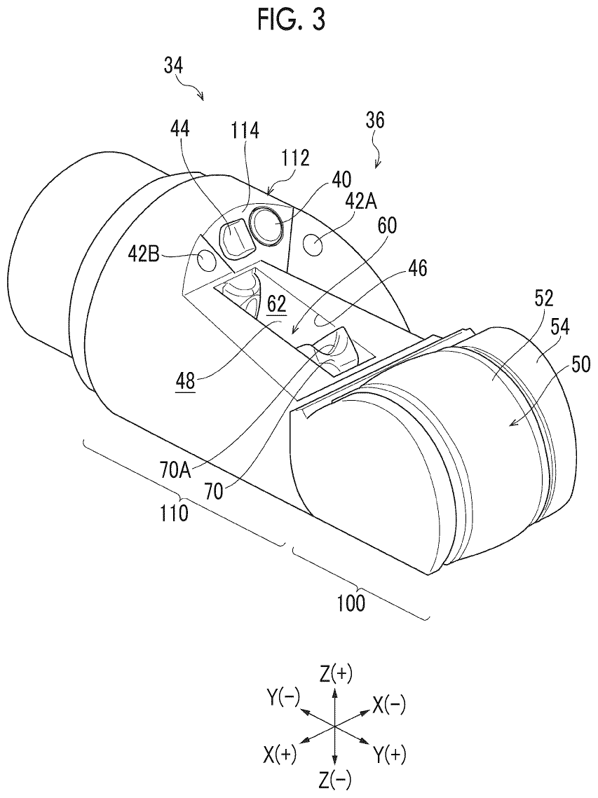

Next, a configuration of the distal end portion 34 (distal end member 36) will be described with reference to FIGS. 2 and 3. FIG. 2 is a bird's-eye view of a front surface side of a distal end member 36 in a case of being viewed obliquely from above. FIG. 3 is a bird's-eye view of a left surface side of the distal end member 36 in a case of being viewed obliquely from above. FIGS. 2 and 3 each show a state in which an elevator 70 to be described later is located at a falling position.

Hereinafter, in a case of describing the configuration of each portion of the distal end member 36, a three-dimensional orthogonal coordinate system of X, Y, and Z will be used for convenience of description. In the drawings, a Z direction indicates an up-down direction, a Z(+) direction side indicates an up direction, and a Z(−) direction side indicates a down direction. In addition, in the drawings, an X direction indicates a direction perpendicular to the Z direction, an X(+) direction side indicates a left direction, and an X(−) direction side indicates a right direction. In addition, in the drawings, a Y direction indicates a direction perpendicular to both the Z direction and the X direction, a Y(+) direction side indicates a distal end side direction, and a Y(−) direction side indicates a base end side direction. It should be noted that each of the above-described directions indicates a direction in a case in which the distal end member 36 is viewed from the distal end side and an ultrasonic wave transmission and reception surface 52 of an ultrasonic transducer 50 described later is directed upward.

In addition, the Y direction corresponds to a longitudinal axis direction of the insertion part according to the embodiment of the present invention (hereinafter, may be simply referred to as a “longitudinal axis direction”). In addition, the Z direction corresponds to a first direction according to the embodiment of the present invention, and the Z(+) direction side corresponds to one side of the first direction according to the embodiment of the present invention.

As shown in FIGS. 2 and 3, the distal end member 36 includes the ultrasonic observation portion 100 and the endoscope observation portion 110 disposed on the Y(−) direction side (base end side) of the ultrasonic observation portion 100. It should be noted that a balloon groove (not shown) to which a balloon that covers the ultrasonic transducer 50 is attached is provided between the ultrasonic observation portion 100 and the endoscope observation portion 110.

The ultrasonic observation portion 100 includes the ultrasonic transducer 50, and the ultrasonic transducer 50 is held by a housing 54.

The ultrasonic transducer 50 is a convex type in which a plurality of ultrasonic oscillators are arranged in an arc shape along the Y direction (longitudinal axis direction). The ultrasonic transducer 50 is configured such that an upper side surface (surface on the Z(+) direction side) thereof is formed as an ultrasonic wave transmission and reception surface 52, and emits an ultrasonic wave from the ultrasonic wave transmission and reception surface 52 toward the Z(+) direction side (one side in the first direction). Specifically, in a case of being viewed in the X direction, the ultrasonic waves emitted from the ultrasonic wave transmission and reception surface 52 (plurality of ultrasonic oscillators) are fan-shaped scanned (convex-scanned) obliquely rearward (Y(−) direction side and Z(+) direction side) to obliquely forward (Y(+) direction side and Z(+) direction side). The emission direction of the ultrasonic waves scanned in this way includes at least a component on the Z(+) direction side. It should be noted that it is sufficient that the emission direction of most of the ultrasonic waves to be scanned include the component on the Z(+) direction side, and the emission direction of some ultrasonic waves need not include the component on the Z(+) direction side (for example, in a case of the Y(+) direction side or in a case in which the direction includes a component on the Z(−) direction side). Data for generating the ultrasonic image is acquired by the ultrasonic transducer 50 configured in this manner.

The endoscope observation portion 110 includes a body member 112 formed in a substantially cylindrical shape. The body member 112 is made of an insulating material having insulating properties, for example, a resin material (base material) such as plastic materials such as a methacrylic resin, a polyphenyl sulfone resin, a polyether imide resin, a polyether ether ketone resin, and a polycarbonate.

The body member 112 includes an elevator housing portion 60 that houses the elevator 70 therein. The elevator housing portion 60 includes an opening 46 having a rectangular shape in plan view in a case of being viewed from the Z(+) direction side. The opening 46 is formed to be open toward the Z(+) direction side (corresponding to “one side in the first direction” according to the embodiment of the present invention) in the body member 112. The opening direction of the opening 46 in the present embodiment is a direction (that is, the Z(+) direction side and the Y(+) direction side) that is obliquely forward in a case of being viewed in the X direction. It should be noted that the opening direction of the opening 46 may be any direction as long as the opening direction is a direction including at least a component on the Z(+) direction side, and may be, for example, a direction including only a component on the Z(+) direction side (that is, a direction perpendicular to the X-Y plane). In other words, a normal direction of a rectangular opening surface 48 (virtual surface) that covers the opening 46 may be a direction including at least the component on the Z(+) direction side. It should be noted that the elevator housing portion 60 is disposed at the distal end portion 34, and is an example of an elevator housing portion according to the embodiment of the present invention.

A treatment tool outlet port (not shown) that communicates with an elevator housing space 62 of the elevator housing portion 60 is provided on the Y(−) direction side (base end side) of the elevator housing portion 60. The treatment tool outlet port communicates with the treatment tool inlet port 24 of the operating part 10 via the treatment tool insertion channel inserted and disposed into the insertion part 12 (see FIG. 1). As a result, the treatment tool introduced from the treatment tool inlet port 24 is guided to the elevator housing space 62 from the treatment tool outlet port via the treatment tool insertion channel.

The elevator 70 is housed in the elevator housing space 62 of the elevator housing portion 60. The elevator 70 is disposed on the Y(−) direction side with respect to the ultrasonic transducer 50. The elevator 70 is provided in a rotationally movable manner about a rotational movement shaft (not shown) disposed along the X direction, and is rotationally moved between an elevating position and a falling position. The elevating position refers to a position of the elevator 70 (that is, a position at which the elevator 70 is in a state of being elevated toward the Z(+) direction side) in a case in which the distal end side (opposite side to the rotational movement shaft) of the elevator 70 is moved to an end position on the Y(−) direction side in a rotationally movable range of the elevator 70. The falling position refers to a position of the elevator 70 (that is, a position at which the elevator 70 is in a state of being fallen toward the Y(+) direction side) in a case in which the distal end side of the elevator 70 is moved to an end position on the Y(+) direction side in the rotationally movable range of the elevator 70.

The elevator 70 is made of a metal material such as stainless steel, and includes a treatment tool guide surface 70A on the upper surface side. The treatment tool guided to the elevator housing portion 60 is guided along the treatment tool guide surface 70A, and is led out of the opening 46 of the elevator housing portion 60. It should be noted that the treatment tool is led out toward an ultrasonic wave scanning range of the ultrasonic transducer 50.

In a case in which the elevating operation lever 18 (see FIG. 1) is operated, the elevator 70 configured as described above operates (elevating and falling operation) between the elevating position and the falling position about the rotational movement shaft. For example, a leading-out direction (leading-out angle) of the treatment tool guided by the treatment tool guide surface 70A of the elevator 70 and led out from the opening 46 can be changed by operating the elevator 70 by the operation of the elevating operation lever 18 and adjusting an elevating angle of the elevator 70 from the falling position.

The body member 112 includes a disposition surface 114 disposed on the Y(−) direction side with respect to the elevator housing portion 60. The disposition surface 114 is a surface on which an air and water supply nozzle 44 and the like, which will be described later, are disposed, and is formed of a surface inclined with respect to the X-Y plane. In other words, in a case of being viewed in the X direction, the disposition surface 114 is formed of surfaces facing obliquely forward (Y(+) direction side and Z(+) direction side). It should be noted that the disposition surface 114 may be formed as a surface perpendicular to the X-Y plane. That is, the disposition surface 114 need only be formed by a surface including at least a normal component toward the Y(+) direction side. It should be noted that the disposition surface 114 is an example of a disposition surface according to the embodiment of the present invention.

The disposition surface 114 is provided with an observation window 40 for observing an inside of a subject, illumination windows 42A and 42B for illuminating the inside of the subject, and an air and water supply nozzle 44 for cleaning the observation window 40 and the like.

Inside each of the illumination window 42A and the illumination window 42B, light emission portions constituting the illumination unit are housed. These light emission portions are connected to a light source device via a light guide. The illumination light transmitted from the light source device through the light guide is emitted from the light emission portions and to irradiate a treatment target part through the illumination window 42A and the illumination window 42B. It should be noted that the illumination window 42A and the illumination window 42B are examples of a first illumination window and a second illumination window according to the embodiment of the present invention.

An imaging system unit including an imaging optical system, a solid-state imaging element, and a circuit board, which constitute an imaging unit (camera unit), is housed in the observation window 40. Accordingly, light (reflected light) from the treatment target part irradiated by the illumination window 42A and the illumination window 42B is taken in from the observation window 40, and the light is imaged as an observation image on the solid-state imaging element through the imaging optical system. Data for generating the endoscopic image is acquired by the imaging unit. It should be noted that the observation window 40 is an example of an observation window according to the embodiment of the present invention.

In a case in which the air and water supply button 20 (see FIG. 1) is operated, the air and water supply nozzle 44 ejects a cleaning liquid such as water or air (fluid) from an ejection port 45 of the air and water supply nozzle 44 toward the observation window 40 or the like. As a result, the observation window 40 or the like is cleaned. It should be noted that the air and water supply nozzle 44 is an example of a fluid nozzle according to the embodiment of the present invention.

Further, in addition to the imaging unit, an ultrasonic cable connected to the ultrasonic transducer 50, an elevator unit, and the like are disposed inside the body member 112. The elevator unit is a unit in which the elevator 70, an elevator support portion that supports the elevator 70 in a rotationally movable manner, and a drive mechanism for rotationally moving the elevator 70 are formed as an integrated component, and the elevator unit constitutes the elevator housing portion 60.

In the field of a medical endoscope, it is desired to reduce the diameter (perform thinning) of the distal end portion of the insertion part, but there is a problem in that it is difficult to reduce the diameter of the distal end portion because the ultrasonic endoscope has many functions. That is, in the ultrasonic endoscope comprising the ultrasonic transducer and the elevator, it is necessary to dispose large built-in components such as the imaging unit, the ultrasonic cable, and the elevator unit in the internal space of the body member 112. Therefore, the distal end portion of the ultrasonic endoscope tends to be increased in diameter.

Therefore, the endoscope 1 according to the present embodiment has the following configuration in order to reduce the diameter of the distal end portion 34.

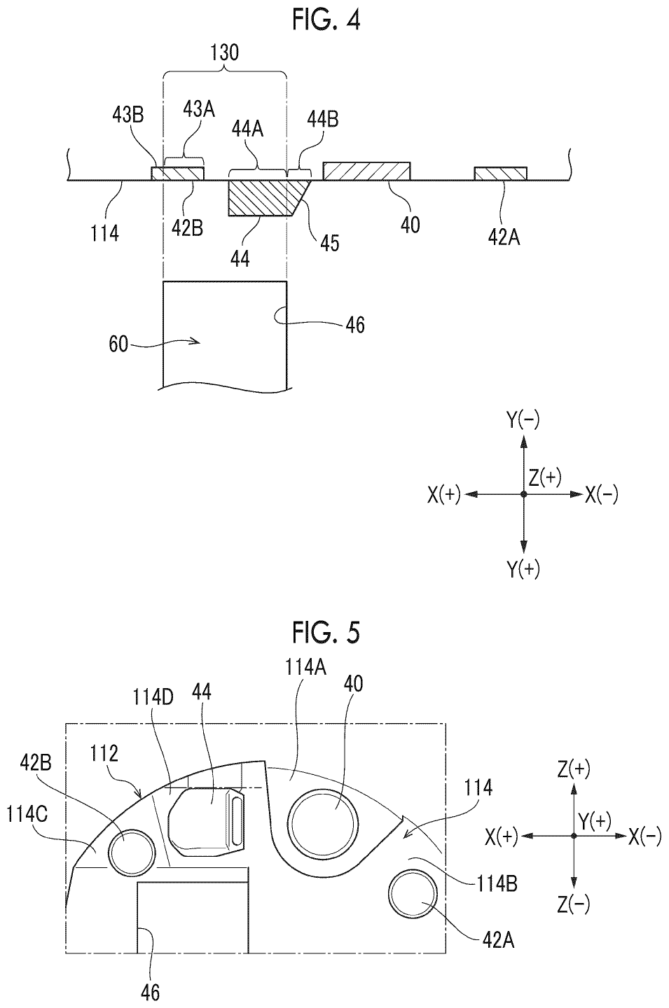

FIG. 4 is an explanatory diagram schematically showing the respective disposition positions of the observation window 40, the illumination window 42A, the illumination window 42B, and the air and water supply nozzle 44 in a case of being viewed from the Z(+) direction side. In addition, in FIGS. 2 and 4, a base end side region 130 in which the opening 46 of the elevator housing portion 60 is extended to the Y(−) direction side is shown. The base end side region 130 is a virtual region, and is an example of the base end side region according to the embodiment of the present invention.

As shown in FIGS. 2 to 4, in the present embodiment, at least a part (preferably, half or more) of a region of the air and water supply nozzle 44 is disposed at a position overlapping with the base end side region 130 in which the opening of the elevator housing portion 60 is extended to the Y(−) direction side in a case of being viewed from the Z(+) direction side (in other words, a side on which the opening 46 is open).

Specifically, a left region 44A of the air and water supply nozzle 44 excluding a right region 44B including the ejection port 45 is disposed at the position overlapping with the base end side region 130. In addition, although not shown, in a more preferred aspect, the air and water supply nozzle 44 may be disposed at a position at which the entire region of the air and water supply nozzle 44 overlaps with the base end side region 130. It should be noted that the left region 44A is an example of at least a part of a region of the fluid nozzle according to the embodiment of the present invention.

Here, in order to reduce the diameter of the distal end portion 34 of the endoscope 1, it is necessary to dispose the light guide and an air and water supply tube, which are the small built-in components, between the large built-in components (the imaging unit, the ultrasonic cable, and the elevator unit). However, since the light guide includes a hard lens barrel, it is difficult to bend the light guide sharply and dispose the light guide. For this reason, a relatively wide disposition space is required to dispose the light guide.

In a configuration of a comparative example, for example, in a case in which all the regions of the illumination window are to be disposed in the base end side region 130 shown in FIG. 4, since a large built-in component (here, the elevator unit is shown) is present in the base end side region 130, a space on the upper side (Z(+) direction side) of the elevator unit needs to be widened in order to secure a disposing space of the light guide. As a result, there is a problem in that the diameter of the distal end portion increases. It should be noted that the same problem occurs even in a case in which the observation window is to be disposed in the base end side region 130.

Therefore, in the present embodiment, as described above, at least a part of a region (left region 44A) of the air and water supply nozzle 44 is disposed at the position overlapping with the base end side region 130. By adopting this configuration, it is possible to dispose the air and water supply tube that only needs to pass water and air and can be sharply bent, and does not require a wide disposition space like the light guide, in a narrow space on the upper side (Z(+) direction side) of the elevator unit. As a result, with the endoscope 1 according to the present embodiment, it is possible to reduce the diameter of the distal end portion 34.

On the other hand, as shown in FIGS. 2 to 4, the observation window 40 is disposed at a position facing the ejection port 45 of the air and water supply nozzle 44 on the disposition surface 114, and the position thereof is on an outer side (X(−) direction side) with respect to the base end side region 130. That is, the observation window 40 is disposed in an outer region on the X(−) direction side of the air and water supply nozzle 44 in an outer region of the base end side region 130.

By disposing the observation window 40 at the above-described position, it is possible to dispose the imaging unit in a relatively spacious disposition space present on the X(−) direction side with respect to the elevator unit. As a result, it is possible to reduce the diameter of the distal end portion 34.

As shown in FIGS. 2 to 4, the illumination window 42A is disposed on the X(−) direction side with respect to the observation window 40 on the disposition surface 114. That is, the illumination window 42A is disposed in the outer region on an opposite side to the side (X(+) direction side) on which the air and water supply nozzle 44 is disposed with respect to the observation window 40 (X(−) direction side), in the outer region of the base end side region 130. The illumination window 42A is disposed on the Z(−) direction side with respect to the observation window 40 in the Z direction.

By disposing the illumination window 42A at the above-described position, it is possible to dispose the light guide of the illumination window 42A in a relatively spacious disposition space present on the X(−) direction side with respect to the elevator unit and the imaging unit. As a result, it is possible to reduce the diameter of the distal end portion 34.

As shown in FIGS. 2 to 4, in the illumination window 42B, at least a part of a region of the illumination window 42B is disposed outside the base end side region 130 on the disposition surface 114. Specifically, a right region 43A on the X(−) direction side of the illumination window 42B is disposed in the base end side region 130, and a left region 43B excluding the right region 43A is disposed outside the base end side region 130 on the X(+) direction side. In addition, in a more preferable aspect, all regions of the illumination window 42B may be disposed an outer side (X(+) direction side) with respect to the base end side region 130, which is not shown in the drawing. That is, in the illumination window 42B, at least a part (left region 43B) of the illumination window 42B is disposed an outer side (X(+) direction side) with respect to the base end side region 130. It should be noted that the left region 43B is an example of at least a part of a region of the second illumination window according to the embodiment of the present invention.

By disposing the illumination window 42B at the above-described position, it is possible to dispose the light guide of the illumination window 42B in a relatively spacious disposition space present on the X(+) direction side with respect to the elevator unit and the imaging unit. As a result, it is possible to reduce the diameter of the distal end portion 34. In addition, since the right region 43A of the illumination window 42B is disposed in the base end side region 130, the treatment target part can be illuminated from the substantially front side by the illumination window 42B.

As described above, in the endoscope 1 according to the embodiment, the two light guides that are difficult to be bent sharply are disposed in the spaces on both sides (X(−) direction side and X(+) direction side) of A relatively spacious base end side region 130 in the X direction, so that the reduction in diameter of the distal end portion 34 can be more effectively achieved. Since the illumination window 42A and the illumination window 42B are disposed on both sides of the observation window 40 in the X direction, the aligning properties are improved.

It should be noted that, in the present embodiment, the endoscope 1 including the two illumination windows 42A and 42B has been described as an example, but the present invention can also be applied to an endoscope including only one of the illumination window 42A or the illumination window 42B.

In the endoscope 1 according to the present embodiment, in a case in which the Z(+) direction side of the Z direction is defined as a height direction, the air and water supply nozzle 44 is disposed at a high position in the distal end member 36 due to a relationship in which the air and water supply nozzle 44 (at least a part thereof) is disposed in the base end side region 130. In this case, since the air and water supply nozzle 44 may come into contact with a mucous membrane in the body cavity, it is desirable to prevent the air and water supply nozzle 44 from coming into direct contact with the mucous membrane and to protect the mucous membrane from the air and water supply nozzle 44.

Hereinafter, some examples (first to fourth examples) of the mucous membrane protection configuration for protecting the mucous membrane from the air and water supply nozzle 44 will be described. It should be noted that, in the following description, the same reference numerals will be assigned to the same or similar members as those shown in FIGS. 1 to 4.

### First Example

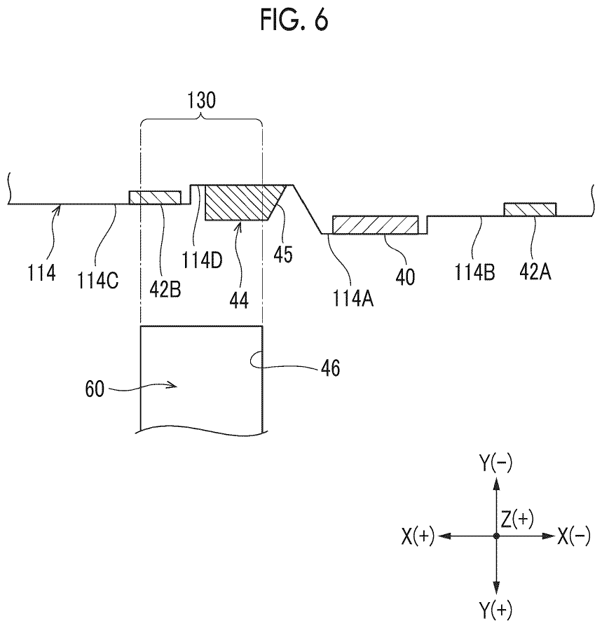

FIG. 5 is an explanatory diagram showing the respective disposition positions of the observation window 40, the two illumination windows 42A and 42B, and the air and water supply nozzle 44 in a case of being viewed from the Y(+) direction side. FIG. 6 is an explanatory diagram schematically showing the respective disposition positions of the observation window 40, the two illumination windows 42A and 42B, and the air and water supply nozzle 44 in a case of being viewed from the Z(+) direction side.

As shown in FIGS. 5 and 6, the disposition surface 114 includes a surface region 114A in which the observation window 40 is disposed, a surface region 114B in which the illumination window 42A is disposed, a surface region 114C in which the illumination window 42B is disposed, and a surface region 114D in which the air and water supply nozzle 44 is disposed.

Here, the heights of the respective surface regions 114A to 114D are compared with each other in a case in which the normal direction of the disposition surface 114 is set as the height direction. It should be noted that the disposition surface 114 is formed of a surface inclined with respect to the X-Y plane as described above, and includes a normal component directed obliquely forward (Y(+) direction side and Z(+) direction side), but, for convenience of description, the direction on the Y(+) direction side in FIG. 6 will be described as the height direction.

As shown in FIG. 6, the heights of the respective surface regions 114A to 114D have a relationship of surface region 114D<surface region 114C<surface region 114B<surface region 114A. That is, in the disposition surface 114 of the present example, the surface region 114D is formed as a surface having a lower height than the surface region 114A. It should be noted that the surface region 114D in which the air and water supply nozzle 44 is disposed and the surface region 114A in which the observation window 40 is disposed are examples of a first surface region and a second surface region according to the embodiment of the present invention, respectively.

With the disposition surface 114 having the surface regions 114A and 114D, the surface region 114D of the air and water supply nozzle 44 is lower than the surface region 114A of the observation window 40, so that it is possible to prevent the air and water supply nozzle 44 from directly coming into contact with the mucous membrane. Accordingly, with the mucous membrane protection configuration of the first example, it is possible to protect the mucous membrane from the air and water supply nozzle 44.

It should be noted that, specifically, the surface region 114D is formed at a position shifted from the surface region 114A to the base end side (Y(−) direction side) by 0.3 mm to 0.5 mm. That is, a level difference is provided between the surface region 114D and the surface region 114A. In this case, it is preferable that the front surface of the air and water supply nozzle 44 is located on the Y(−) direction side with respect to the front surface of the observation window 40 for each front surface of the air and water supply nozzle 44 and the observation window 40 facing the Y(+) direction side. As a result, it is possible to further suppress the direct contact of the air and water supply nozzle 44 with the mucous membrane.

### Second Example

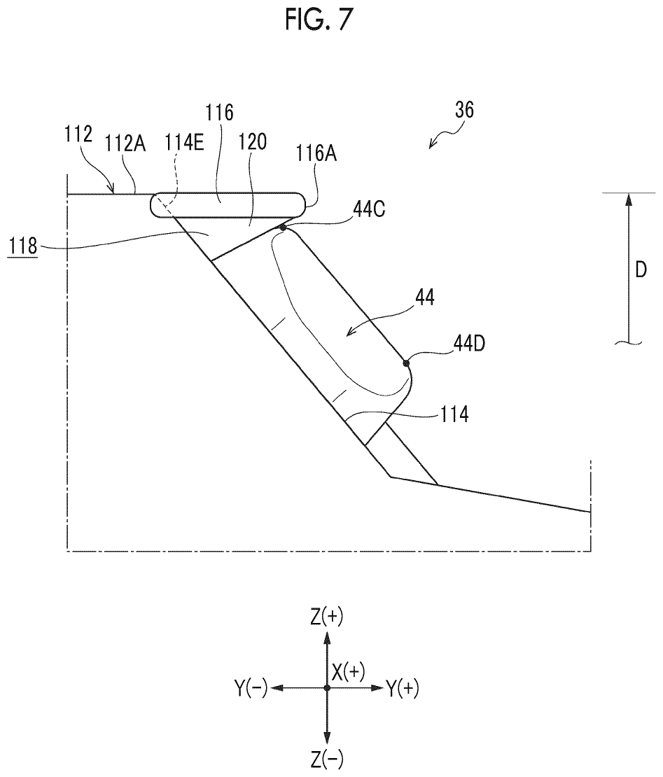

FIG. 7 is an enlarged view of the air and water supply nozzle 44 disposed on the disposition surface 114 in a case of being viewed from the X(+) direction side.

As shown in FIG. 7, the distal end member 36 comprises a cover member 116. The cover member 116 is disposed in a surface region 114E that is located on the Z(+) direction side (one side) of the air and water supply nozzle 44 in the Z direction (first direction) on the disposition surface 114 of the body member 112. It should be noted that the cover member 116 is an example of a cover member according to the embodiment of the present invention.

In addition, the cover member 116 is provided at a position that is not beyond an outer diameter D (diameter) of the body member 112 in the surface region 114E. Specifically, in a case in which the Z(+) direction side is defined as the height direction, the cover member 116 is provided at the same height position as an upper surface 112A of the body member 112 or at a position lower than the upper surface 112A.

In addition, the cover member 116 is provided to protrude from the surface region 114E to the Y(+) direction side, and the cover member 116 covers a most upper end portion 44C on the Z(+) direction side of the air and water supply nozzle 44. Therefore, in a case of being viewed from the Z(+) direction side, the upper end portion 44C of the air and water supply nozzle 44 is hidden by the cover member 116.

By providing the cover member 116 in the body member 112, it is possible to prevent the upper end portion 44C of the air and water supply nozzle 44 from being in direct contact with the mucous membrane. Accordingly, with the mucous membrane protection configuration of the second example, it is possible to protect the mucous membrane from the air and water supply nozzle 44.

It is preferable that the cover member 116 is made of the same material (resin material) as the body member 112. As a result, the body member 112 and the cover member 116 can be integrally formed (integrally molded product).

By forming the distal end portion 116A of the cover member 116 on the Y(+) direction side in an R-shape (curved shape) as shown in FIG. 7, it is possible to relieve the stimulus applied from the distal end portion 116A to the mucous membrane.

In addition, since a filler 120 that fills a gap 118 between the air and water supply nozzle 44 and the cover member 116 is provided, in a case in which the cover member 116 comes into contact with the mucous membrane, the filler 120 serves as a buffer member that absorbs a force received from the cover member 116, so that the stimulus applied to the mucous membrane from the cover member 116 can be relieved. It should be noted that the filler 120 is an example of a first filler according to the embodiment of the present invention.

It should be noted that, in the second example of FIG. 7, the configuration has been described in which only the upper end portion 44C of the air and water supply nozzle 44 is covered by the cover member 116, but the present invention is not limited to this. For example, the distal end portion 116A of the cover member 116 may be further extended to the Y(+) direction side, and the distal end portion 116A may cover the lower end portion 44D of the air and water supply nozzle 44 in a case of being viewed from the Z(+) direction side.

### Third Example

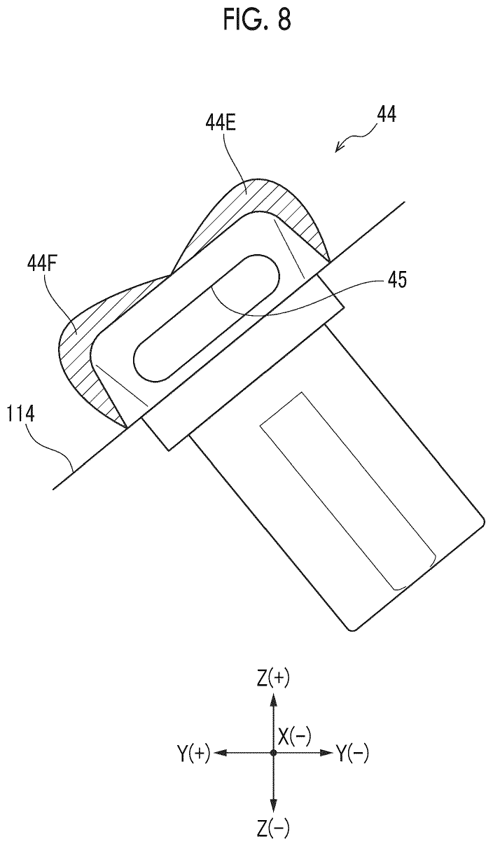

FIG. 8 is an enlarged view of the air and water supply nozzle 44 disposed on the disposition surface 114 in a case of being viewed from the X(−) direction side.

As shown in FIG. 8, in a case in which the air and water supply nozzle 44 is viewed from the side of the ejection port 45 (X(−) direction side) of the air and water supply nozzle 44, a first end portion 44E and a second end portion 44F forming the edge portion of the air and water supply nozzle 44 are formed in an R-shape (curved shape).

With the air and water supply nozzle 44 having the R-shape, in a case in which the air and water supply nozzle 44 comes into contact with the mucous membrane, it is possible to relieve the stimulus applied from the air and water supply nozzle 44 to the mucous membrane. Accordingly, with the mucous membrane protection configuration of the third example, it is possible to protect the mucous membrane from the air and water supply nozzle 44.

It should be noted that, in the above-described air and water supply nozzle 44, both the first end portion 44E side and the second end portion 44F side have an R-shape, but only the first end portion 44E, which is the upper end portion on the Z(+) direction side that is likely to come into contact with the mucous membrane, may have an R-shape. However, since the second end portion 44F side also has an R-shape, it is possible to protect the mucous membrane in a case in which the second end portion 44F comes into contact with the mucous membrane. In addition, it is preferable that the first end portion 44E and the second end portion 44F having an R-shape are made of the same material as the air and water supply nozzle 44 in terms of integrally molding, but the first end portion 44E and the second end portion 44F may be made of different materials and may be attached later.

### Fourth Example

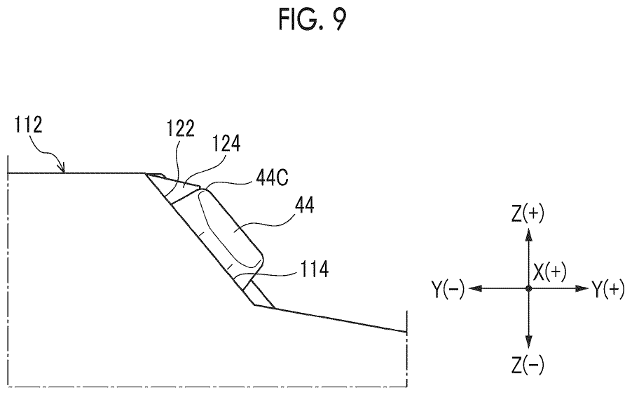

FIG. 9 is an enlarged view of the air and water supply nozzle 44 disposed on the disposition surface 114 in a case of being viewed from the X(+) direction side.

As shown in FIG. 9, the body member 112 comprises a filler 124 that fills a level difference 122 formed on the Z(+) direction side of the air and water supply nozzle 44. It should be noted that the filler 124 is an example of a second filler according to the embodiment of the present invention.

By filling the above-described level difference 122 with the filler 124, the edge portion of the upper end portion 44C of the air and water supply nozzle 44 on the Z(+) direction side can be hidden by the filler 124, so that, in a case in which the air and water supply nozzle 44 comes into contact with the mucous membrane, the stimulus applied from the air and water supply nozzle 44 to the mucous membrane can be relieved. Accordingly, with the mucous membrane protection configuration of the fourth example, it is possible to protect the mucous membrane from the air and water supply nozzle 44.

It should be noted that, in a case in which the Z(+) direction side is defined as the height direction, the filler 124 is provided at the same height position as the upper surface 112A of the body member 112 or at a lower position than the upper surface 112A from the viewpoint of achieving the reduction in diameter of the distal end portion 34.

In the process of performing the procedure using the ultrasonic endoscope, a contrast medium and the like may adhere to the periphery of the elevator. In a case in which the contrast medium is solidified in the elevator, there is a problem in that the insertion resistance of the treatment tool is larger than the initial state.

In the configuration of the distal end portion 34 shown in FIGS. 2 and 3, the cleaning water ejected from the ejection port 45 of the air and water supply nozzle 44 flows toward the observation window 40 on the X(−) direction side. For this reason, the cleaning water may not be sufficiently supplied to the elevator 70 located on the Z(−) direction side of the air and water supply nozzle 44.

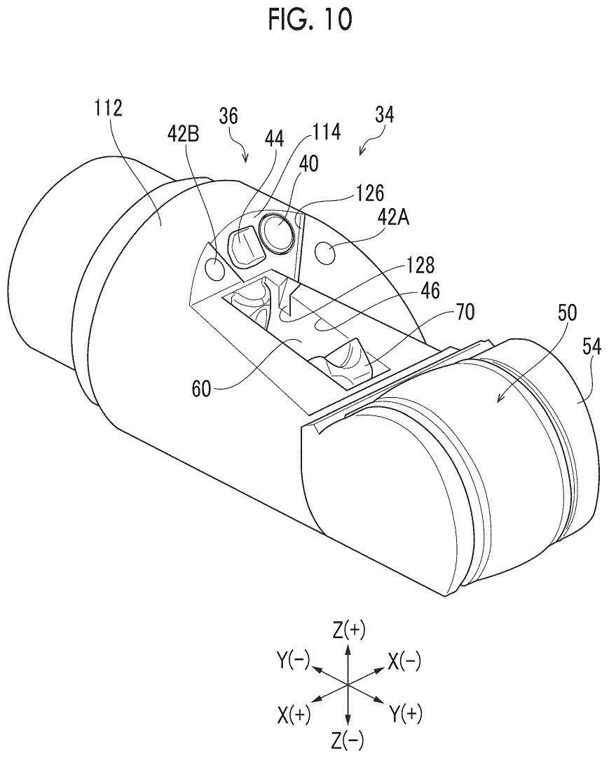

Therefore, FIG. 10 shows the distal end portion 34 having a cleaning structure for effectively washing away the contrast medium adhering to the elevator 70 with the cleaning water ejected from the ejection port 45 of the air and water supply nozzle 44.

With the distal end portion 34 shown in FIG. 10, the guide surface 126 is provided on the disposition surface 114 of the body member 112. The guide surface 126 is a surface that guides the cleaning water ejected from the ejection port 45 toward the elevator housing portion 60, and is provided at a position on the opposite side (X(−) direction side) to a side on which the air and water supply nozzle 44 is provided with respect to the observation window 40. That is, the guide surface 126 is a level difference surface provided at a position facing the ejection port 45 side of the air and water supply nozzle 44 with the observation window 40 interposed therebetween, and reflects and guides the cleaning water ejected from the ejection port 45 toward the elevator housing portion 60.

With the distal end portion 34 including such a guide surface 126, the cleaning water ejected from the ejection port 45 of the air and water supply nozzle 44 passes through the surface of the observation window 40, collides with the guide surface 126, and then is reflected and guided toward the elevator housing portion 60 by the guide surface 126. Accordingly, the cleaning water is supplied to the elevator housing portion 60, and the contrast medium adhering to the elevator 70 can be washed away with the cleaning water.

As shown in FIG. 10, it is preferable that the distal end portion 34 is provided with a guide groove 128 on an edge portion of the opening 46 of the elevator housing portion 60. The guide groove 128 is formed of a cutout groove in which a part of the edge portion of the opening 46 of the elevator housing portion 60 is cut out.

With the distal end portion 34 including such a guide groove 128, the cleaning water guided by the guide surface 126 toward the elevator housing portion 60 flows through the guide groove 128 and is supplied to the elevator housing portion 60. As a result, the contrast medium adhering to the elevator 70 can be efficiently washed away with the cleaning water.

Although the ultrasonic endoscope according to the present embodiment has been described above, the present invention may be improved or modified in some ways without departing from the gist of the present invention.

### Explanation of References

1: ultrasonic endoscope
10: operating part
12: insertion part
14: universal cord
16: angle knob
18: elevating operation lever
20: air and water supply button
22: suction button
24: treatment tool inlet port
30: soft portion
32: bendable portion
34: distal end portion
36: distal end member
40: observation window
42A: illumination window
42B: illumination window
43A: right region
43B: left region
44: air and water supply nozzle
44A: left region
44B: right region
44C: upper end portion
44D: lower end portion
44E: first end portion
44F: second end portion
45: ejection port
46: opening
48: opening surface
50: ultrasonic transducer
52: ultrasonic wave transmission and reception surface
54: housing
60: elevator housing portion
62: elevator housing space
70: elevator
70A: treatment tool guide surface
100: ultrasonic observation portion
110: endoscope observation portion
102: balloon groove
112: body member
112A: upper surface
114: disposition surface
114A: surface region
114B: surface region
114C: surface region
114D: surface region
114E: surface region
116: cover member
116A: distal end portion
118: gap
120: filler
122: level difference
124: filler
126: guide surface
128: guide groove
130: base end side region

What is claimed is:

1. An ultrasonic endoscope comprising:
a distal end portion that is disposed on a distal end side of an insertion part extending along a longitudinal axis direction;
an elevator housing portion that is disposed at the distal end portion, that has an opening on one side in a first direction orthogonal to the longitudinal axis direction, and that houses an elevator therein; and
a disposition surface that is disposed on a base end side in the longitudinal axis direction with respect to the elevator housing portion, that includes a normal component toward a distal end side in the longitudinal axis direction, and that is provided with a fluid nozzle and an observation window disposed thereon,
wherein, in a case of being viewed from the one side in the first direction, at least a part of a region of the fluid nozzle is disposed at a position overlapping with a base end side region in which the opening of the elevator housing portion is extended to the base end side in the longitudinal axis direction.

2. The ultrasonic endoscope according to claim 1,
wherein the observation window is disposed outside the base end side region.

3. The ultrasonic endoscope according to claim 2,
wherein the observation window is disposed at a position facing an ejection port side of the fluid nozzle.

4. The ultrasonic endoscope according to claim 1,
wherein the disposition surface is provided with a first illumination window disposed outside the base end side region.

5. The ultrasonic endoscope according to claim 4,
wherein the first illumination window is disposed on an opposite side to a side on which the fluid nozzle is disposed with respect to the observation window.

6. The ultrasonic endoscope according to claim 1,
wherein the disposition surface is provided with a second illumination window of which at least a part of a region is disposed outside the base end side region.

7. The ultrasonic endoscope according to claim 6,
wherein the second illumination window is disposed on an opposite side to a side on which the observation window is disposed with respect to the fluid nozzle.

8. The ultrasonic endoscope according to claim 1,
wherein the disposition surface includes a first surface region in which the fluid nozzle is disposed and a second surface region in which the observation window is disposed, and
in a case in which a normal direction of the disposition surface is defined as a height direction, a height of the first surface region is lower than a height of the second surface region.

9. The ultrasonic endoscope according to claim 1, further comprising:
a cover member that is provided at the distal end portion and that is disposed on one side of the fluid nozzle in the first direction.

10. The ultrasonic endoscope according to claim 9,
wherein the cover member is made of a material that is the same as a material of the distal end portion.

11. The ultrasonic endoscope according to claim 9,
wherein the cover member covers an outermost end portion side of the fluid nozzle on the one side in the first direction.

12. The ultrasonic endoscope according to claim 9, further comprising:
a first filler that fills a gap between the fluid nozzle and the cover member.

13. The ultrasonic endoscope according to claim 1,
wherein, in a case of being viewed from an ejection port side of the fluid nozzle, an outermost end portion side of the fluid nozzle on the one side in the first direction is formed in an R-shape.

14. The ultrasonic endoscope according to claim 1, further comprising:
a second filler that fills a level difference formed on one side of the fluid nozzle in the first direction.

15. The ultrasonic endoscope according to claim 1,
wherein the disposition surface includes a guide surface that is provided on an opposite side to a side on which the fluid nozzle is provided with respect to the observation window and that guides fluid ejected from the fluid nozzle toward the elevator housing portion.

16. The ultrasonic endoscope according to claim 15,
wherein a guide groove that guides the fluid guided by the guide surface to an inside of the elevator housing portion is provided at an edge portion of the opening of the elevator housing portion.
